(19) United States
(12) Patent Application Publication
(10) Pub. No.: US 2018/0021385 A1
(43) Pub. Date: Jan. 25, 2018

(54) WOUND HEALING FORMULATION

(71) Applicants: Stichting VUmc, Amsterdam (NL); A-Skin Holding B.V., Amsterdam (NL)

(72) Inventors: Susan GIBBS, Amsterdam (NL); Riekeld Johannes SCHEPER, Amsterdam (NL)

(21) Appl. No.: 15/548,666

(22) PCT Filed: Dec. 22, 2015
(86) PCT No.: PCT/NL2015/050901

§ 371 Date: Aug. 3, 2017

(30) Foreign Application Priority Data
Feb. 4, 2015 (NL) 2014230

(51) Int. Cl. A61K 35/36; C12N 5/071; C12N 5/077; A61K 35/33

(52) U.S. Cl. CPC A61K 35/36; A61K 35/33; C12N 5/0629; C12N 5/0656; C12N 2502/094; C12N 2502/1323

(57) ABSTRACT

The present disclosure relates to a method for producing a composition, wherein the method comprises the steps of co-culturing immortalized fibroblasts and immortalized keratinocytes, thereby producing secretion; separating the secretion from the fibroblasts and keratinocytes; and providing a pharmaceutically acceptable composition comprising the secretion. The present disclosure also relates to the composition obtainable by the method, wherein the composition preferably is a pharmaceutical composition for medical use, preferably for use in the treatment of a wound, preferably a chronic or acute wound.

### CROSS-REFERENCE TO RELATED APPLICATION

This application is the National Stage of International Application No. PCT/NL2015/050901, filed 22 Dec. 2015, which claims the benefit of and priority to NL Application No. 2014230, having the title “Wound Healing Formulation,” filed on 4 Feb. 2015, the entire disclosures of which are incorporated by reference in their entireties as if fully set forth herein.

### TECHNICAL FIELD

The present disclosure relates to the field of wound healing therapies, in particular for treating acute or chronic wounds.

### STATE OF THE ART

Chronic and difficult-to-heal wounds represent a considerable challenge to medical practitioners. For example, ulcers (diabetic, decubitus, venous/arterial origin) can exist for many years. 5-10% of these ulcers are highly therapy resistant and unresponsive to local therapy, compression therapy (venous origin) vacuum assisted closure therapy and autografts. Also surgical wounds created for example by excision of malignancies can degenerate to difficult to heal wounds. Furthermore, burn patients can develop large acute as well as chronic wounds which are difficult to close using standard methods.

Therefore, the development of improved wound healing therapies is a continuing goal. For example, Blok et al discuss the safety and efficacy of autologous dermal-epidermal skin substitutes (SS) for treating ulcers of various origins (Blok et al. 2013, Wound Repair and Regeneration, 21: 667-676)

It is an object of the present disclosure to improve and/or complement wound healing therapies, in particular for treating chronic wounds, but also for treating acute wounds.

### SUMMARY OF THE PRESENT DISCLOSURE

The present disclosure relates to a method for the preparation of a composition with wound healing properties, wherein the method comprises the steps of a) co-culturing immortalized fibroblasts and immortalized keratinocytes, thereby producing secretion containing wound healing mediators; b) separating the secretion from the fibroblasts and keratinocytes; and preferably c) providing a pharmaceutically acceptable composition comprising the secretion.

In this way, a very potent wound healing formulation (i.e. the secretion) can be obtained which can be used for the treatment of a wound, such as a skin wound, an acute or chronic wound, an ulcer or a decubitus wound (i.e. pressure sore).

Before the present disclosure was made, it was accepted practice in the art that primary cells (cells derived from e.g. the patient) should be used for obtaining secreted wound healing mediators. Preferably those primary cells were comprised in a skin construct resembling natural skin. The belief was that the natural situation and the natural cellular interactions should be mimicked as closely as possible in order to come to potent wound healing formulations.

For example, WO01/14527 discloses a method for producing a wound healing composition by using a skin construct that is derived from donor tissue. WO01/14527 thus does not apply immortalized cells as according to the present invention. As another example, Spiekstra et al suggest that full-skin substitutes have the greatest potential in stimulating wound healing (Spiekstra et al 2007, Wound Rep Reg 15: 708-717).

General Definitions

In the following description and examples, a number of terms are used. In order to provide a clear and consistent understanding of the description and claims, including the scope to be given such terms, the following definitions are provided. Unless otherwise defined herein, all technical and scientific terms used have the same meaning as commonly understood by one of ordinary skill in the art to which this disclosure belongs.

The term “wound” refers to an opening in skin tissue or mucosal tissue, i.e. to a skin or mucosal defect. Non-limiting examples of such wound are burn wounds, ulcers, surgical excision wounds, wounds created by reconstruction surgery and wounds caused by release of contractures as a result of hypertrophic scarring. In one embodiment, such wound is a chronic wound, such as an (inert) ulcer. In another embodiment, such wound is an acute wound, such as a burn wound, surgical excision wound, a wound created by reconstruction surgery or a wound caused by release of contractures as a result of hypertrophic scarring. Typically, a wound is a type of physical trauma where the integrity of the skin or tissue is disrupted. If the outer layer of a tissue is damaged the wound is considered an open wound. Wounds that do not heal within 2 or 3 months are considered chronic. An ulcer is discontinuity of the skin or mucous membrane, typically a crater-like sore. Ulcers can form when outer layers of skin or tissue have been removed. They can occur on the skin, in the mouth, stomach, and other parts of the body. Acute wounds are new wounds (e.g. less than 1, 3, 7, or 15 days old) and include surgical incisions and traumatic injuries such as lacerations, abrasions, avulsions, penetrations or bites, and burn injuries. Acute wounds normally proceed through an orderly and timely reparative process that results in sustained restoration of anatomic and functional integrity.

The term “treatment of a wound” refers to application of the composition according to the present disclosure onto the wound as to stimulate wound healing. Such treatment may be carried out as sole therapy, or may be used as a pre-treatment to prepare the wound for the application of further wound closing and healing techniques such as compression therapy, vacuum persistent closure or application of a tissue substitute or an autograft. The composition may also be used for combination therapy, e.g. in combination with one or more other wound closing and healing techniques.

The term “keratinocyte” refers to a type of cell, preferably human, that synthesizes keratin. In vivo, keratinocyte cells are the main constitute of the epidermis and are formed from undifferentiated cells at the dermal-epidermal junction. A characteristic filament protein of keratinocytes is cytokeratin. Keratinocytes typically can be identified by showing staining for cytokeratin, for example by immunohistochemistry using an antibody against cytokeratin such as M3515 obtainable from Dako. At the same time staining for vimentin typically will be negative, for example by immunohistochemistry using an antibody against vimentin such as M0725 obtainable from Dako. Furthermore, in appropriate media, keratinocytes may be able to produce IL-1α. Also, or alternatively, a keratinocyte can be identified if recognized as such by a skilled person, based for example on structural or functional features. In the context of the present disclosure, a keratinocyte may be immortalized.

The term “fibroblast” refers to a type of cell, preferably human, that synthesizes extracellular matrix and collagen. Fibroblasts are the most common cells of connective tissue in humans. Vimentin is the most frequently found intermediate filament in fibroblasts. Thus, fibroblasts may be identified by staining for vimentin, for example by immunohistochemistry using an antibody against vimentin such as M0725 obtainable from Dako. Furthermore or alternatively, fibroblasts can be identified by producing CXCL-8/IL-8 in response to (recombinant) IL-1α. Fibroblasts are morphologically heterogeneous with diverse appearances depending on their location and activity. Nonetheless, a fibroblast can be identified if recognized as such by a skilled person, based for example on structural or functional features. In the context of the present disclosure, a fibroblast may be immortalized.

The term “immortalized” in the context of the present disclosure means a cell which is capable of being passaged many more times than the original cells, e.g. 10, 20, 50 or more times. Passage number refers to the number of times the cell line has been re-plated and grown back to confluency. Maximum passage number of primary keratinocytes while maintaining their quality and functionality typically is 2, and of primary fibroblasts typically 2-4. BJ-5ta immortalized fibroblasts (obtained from ATCC at unknown passage number Px) have for example already been shown to be able to be passaged for at least Px+50. A431 immortalized keratinocytes (obtained at P29) are spontaneously immortalized (epidermoid carcinoma) and have for example been shown to be able to be passaged for at least P29+62. As will be clear, the term “immortalized” in the context of the present disclosure does not require that passage number capability is actively increased, since it can also include for example immortalized cells derived from a spontaneous carcinoma. However, apart from isolation from e.g. carcinoma, cells can also be actively immortalized by methods known to those skilled person. For example, lengthening of the lifespan of a cell can be achieved by the transfer of a virus or a plasmid that contains one or more immortalizing genes. Immortalizing genes are well known to the skilled person. See, e.g., Katakura et al., Methods Cell Biol. 57:69-91 (1998). Immortalizing proteins or polypeptides include e.g. 12S and 13S products of the adenovirus E1A genes, SV40 small and large T antigens, papilloma viruses E6 and E7, the Epstein-Barr Virus (EBV), Epstein-Barr nuclear antigen-2 (EBNA2), human T-cell leukemia virus-I (HTLV-1), HTLV-1 tax, Herpesvirus Saimiri (HVS), mutant p53, and the proteins from oncogenes such as myc, c-jun, c-ras, c-Ha-ras, h-ras, v-src, c-fgr, myb, c-myc, n-myc, and Mdm2. For the present disclosure, a preferred immortalization strategy is transformation of the cell with the gene encoding telomerase reverse transcriptase (TERT) such that TERT is either stably or transiently expressed thereby resulting in the expression of telomerase activity. Telomerase activity can lead to elongation of the chromosome tips or protective caps, called telomeres, thereby resulting in the ability to become immortalized without becoming transformed (See Jiang, et al., Nature Genetics 21:111-14 (1999) and Morales, et al., Nature Genetics 21:115-18 (1999)). The hTERT immortalisation method as described in Lee et al Cytotechnology. June 2004; 45(1-2): 33-38 is also particularly preferred. A number of well-known methods exist for introducing genetic material into target cells. These include the use of polycations such as DEAE-dextran (see McCutchan, et al., J. Natl. Cancer Inst. 41:351-57 (1968) and Kawai et al., Mol. Cell. Biol. 4:1172-74 (1984)); calcium phosphate coprecipitation (see Graham et al., Virology 52:456-67 (1973)); electroporation (see Neumann et al, EMBO J. 7:841-45 (1982)); lipofection (see Feigner et al., Proc. Natl. Acad. Sci. USA 84:7413-17 (1987)); retrovirus vectors (see Cepko et al., Cell 37:1053-62 (1984)); and microinjection (see Capecchi et al., Cell 22:479-88 (1980)). Alternatively, a transient immortalization using a protein domain transport sequence (TAT, VP22, MTS, etc.) attached to the TERT protein can also be preferable because then the gene is not permanently inserted but is instead added as a fusion protein to the growth medium.

The term “co-culturing” or “co-culture” refers to the culturing (or growth, maintenance) of two (or more) different cell types in a combined culture, i.e. present in the same container and/or consuming the same culture medium. For example, keratinocytes and fibroblasts may be cultured in vitro, e.g. together in one container. In vitro means outside a human or animal body, or outside the environment where the cells occur naturally. Typically, the cells in the co-culture are randomly organized (or intimately mixed) and/or may be arranged in a monolayer or bilayer (having a thickness corresponding to 1-2 cells). The cells may thus for example be in a different organization as compared to the situation in natural or substitute skin. For example, keratinocytes and fibroblasts can be arranged together in a monolayer, e.g. on a culture plate. The keratinocytes and fibroblasts can be placed in the vicinity of each other such as to allow (chemical) interaction.

The term “secretion” refers to (the totality of) organic molecules and inorganic elements secreted by a cell, or by a co-culture of cells, for example a co-culture of keratinocytes and fibroblasts. The secretion can be an (aqueous) solution or (clinical grade) medium comprising said organic molecules and inorganic elements, preferably being or including CCL-2; CXCL-1; CXCL-8; CCL-5; IL-6; IL-1α; TIMP-2; VEGF; and HGF. In the context of the present disclosure, the term “secretion” is interchangeable with the term “secretome”. The concept of secretome is useful as it can refer to all the secreted compounds.

The terms “CCL-2”, “MCP-1”, “CXCL-1”, “GROa”, “CXCL-8”, “IL-8”, “CCL-5”, and “RANTES” refer to specific chemokines. Chemokines typically are small, secreted pro-inflammatory proteins, which are responsible for attracting infiltrating immune cells into a wound bed in order to fight infection and remove damaged tissue. Increasing evidence shows that chemokines are also responsible for attracting skin residential cells (epidermal cells, fibroblasts and endothelial cells) into the wound bed. In other words, chemokines are signaling proteins which act as a chemoattractant to guide migration of cells. Cells that are attracted by chemokines typically follow a signal of increasing chemokine concentration towards the source of the chemokine. Chemokines are functionally divided into two groups: (1) homeostatic and (2) inflammatory. CCL-2/MCP-1; CXCL-1/GROα; CXCL-8/IL-8; and CCL-5/RANTES are examples of inflammatory chemokines. CCL-2/MCP-1; CXCL-1/GROα; and CCL-5/RANTES can be quantified by ELISA measurements using specific antibodies which can be obtained for example from R&D Systems Inc. (Minneapolis, Minn., USA). CXCL-8/IL-8 can for example be quantified using a Pelipair reagent set obtainable from Sanquin (Amsterdam, the Netherlands).

The terms “IL-6”, “IL-1α”, and “TNF-α” refer to specific cytokines. Cytokines are a category of proteins, typically ˜5-20 kDa, that are important in cell signaling. Cytokines are secreted by cells and can affect behavior of cells, such as cell migration. Interleukins IL-6 and IL-1α are involved in the regulation of immune responses. The primary role of TNF-α is in the regulation of immune cells. IL-6 and IL-1α can be quantified by ELISA measurements using specific antibodies which can be obtained for example from R&D Systems Inc. (Minneapolis, Minn., USA). TNF-α can for example be quantified using a Pelipair reagent set obtainable from Sanquin (Amsterdam, the Netherlands).

The terms “TIMP-2”, “VEGF”, and “HGF” refer to specific proteases (inhibitors) and growth factors. TIMP-2 is a member of tissue inhibitors of metalloproteinases (TIMPs). Vascular endothelial growth factor (VEGF) is a signal protein produced by cells that in vivo stimulates vasculogenesis and angiogenesis. Hepatocyte growth factor (HGF) is a cellular growth factor. TIMP-2, VEGF, and HGF can be quantified by ELISA measurements using specific antibodies which can be obtained for example from R&D Systems Inc. (Minneapolis, Minn., USA).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DETAILED DESCRIPTION OF THE DISCLOSURE

The present disclosure relates to a method for producing a composition, wherein the method comprises the steps of:
a) co-culturing immortalized fibroblasts and immortalized keratinocytes, thereby producing secretion;
b) separating the secretion from the fibroblasts and keratinocytes';
c) preferably providing a pharmaceutically acceptable composition comprising the secretion.

In this way, a secretion can be obtained that is surprisingly rich in wound healing mediators. In view thereof, the secretion is an ideal composition for treating a wound.

Another advantage is that the production of the composition is not dependent on donor availability and donor variation, because no donor tissue is necessary. Furthermore, since the secretion is separated from the cells, a reduced risk of rejection of foreign cells is incurred as compared to a situation wherein such cells are applied to the wound that is to be treated. Moreover, the composition can be applied to wounds of different patients more uniformly and predictably, since it is less dependent on conditions within the wound site as compared to the situation wherein cells are applied to the wound which interact with said conditions. In addition, production of the composition can be up-scaled more easily, since the production of wound healing mediators no longer takes place at the wound site, but instead e.g. in a bioreactor under ideal circumstances.

As the skilled person will understand, step a) can be performed such that the co-cultured cells produce secretion which can be obtained accordingly, i.e. under conditions allowing so. To such aim, step a) can be performed in a medium and temperature allowing the production of secretion. In principle, all commercially available basal media are suitable which provide the basic nutrient source for cells in the form of glucose, amino acids, vitamins, and inorganic ions, together with other basic media components (preferably calcium free).

Clinical grade medium (i.e. medium according to cGMP) such as DMEM/HamF12 medium, e.g. in a 3-to-1 ratio (obtainable from e.g. Sigma Aldrich) is particularly preferred, while research medium is not preferred because it is less or not eligible for human application. Furthermore, Step a) can be performed preferably at a temperature of 30-40° C., preferably 34-38° C. and/or for at least 1, 12, 20 and/or at most 28, 48, 240 hours. It will also be clear that step a), or even the entire method of the present disclosure may be performed in vitro.

In a preferred embodiment of the method according to the present disclosure, a specific ratio between the immortalized fibroblasts and the immortalized keratinocytes is used at the start of the co-culturing period, namely 75%/25% (both ±10%). Particularly, the percentage of the immortalized fibroblasts relative to the total number of immortalized fibroblasts and immortalized keratinocytes can be between 5% and 95%, preferably between 25% and 95%, more preferably between 50% and 95%, more preferably between 65% and 95%, most preferably between 65% and 85%. Accordingly, the percentage of the immortalized keratinocytes relative to the total number of immortalized fibroblasts and immortalized keratinocytes at the start of co-culturing can be between 5% and 95%, preferably between 5% and 75%, more preferably between 5% and 50%, more preferably between 5% and 35%, most preferably between 15% and 35%.

By using the preferred ratios, a wound healing formulation can be obtained that is even richer in wound healing mediators as compared to for example 50%/50% ratio. In principle, the amount of fibroblasts and/or keratinocytes in the co-culture can be between 10³ and 10⁸ cells per mL of medium, preferably between 10⁴ and 10⁷ cells per mL of medium, more preferably between 10⁵ and 5×10⁶ cells per mL of medium. The medium including the wound healing mediators (but separated from the cells) can be readily used for wound healing purposes.

Although any (commercially) available or provided fibroblast cells and/or keratinocyte cells can be used in the method as long as they are immortalized, it may be particularly advantageous to use TERT-immortalized fibroblasts (e.g. BJ-5ta obtainable from ATCC, CRL-4001); and/or A431 keratinocytes (obtainable from ATCC, CRL-1555) or TERT-immortalized keratinocytes (e.g. N/TERT-1 obtainable from Rheinwald's lab), in order to further improve wound healing potency of the wound healing formulation as compared to the use of other combinations of immortalized fibroblasts and keratinocytes.

The culturing of the cells in step a) is preferably performed such that the cells in the co-culture are randomly organized and/or arranged in a monolayer or bilayer, or are in a different organization as compared to the situation in natural or substitute skin. For example, in step a), no extracellular matrix and/or no collagen (and/or no fibrin) is present, or less than 1 wt. % extracellular matrix and/or collagen (and/or fibrin) is present relative to the weight of the coculture (including medium);
no (differentiated) epidermal layer e.g. comprising a stratum basale, stratum spinosum, stratum granulosum and/or stratum corneum is present; and/or
no dermal layer e.g. comprising extracellular matrix is present.

The above means for example that a haematoxylin-eosin staining of the co-culture would not show a stratified epidermal layer with a stratum basale, stratum spinosum, stratum granulosum and stratum corneum. Also no distinct dermal layer would be shown in such staining. It is also preferred that no other cell types are present in the co-culture, or less than 1% as compared to the total number of cells.

Also the following materials are preferably not present in step a) of the method, or less than 1 wt. % relative to the weight of the coculture: collagens, alginate, alginate beads, agarose, fibrin, fibrin glue, blood plasma fibrin beads, whole plasma or components thereof, laminins, fibronectins, proteoglycans, HSP, chitosan, heparin, and/or other synthetic polymer scaffolds and solid support materials that could hold or adhere to cells such as wound dressings.

In step b), in order to separate the secretion from the fibroblasts and keratinocytes, centrifugation and/or filtering with a (0.22 μm) filter can be applied, wherein preferably filtering is applied on supernatant, i.e. after separating most cells from the secretion by centrifugation. Filtering appeared to have no effect on the protein concentration, particularly when filtering is performed before storage (direct filtering).

Step b) can thus be performed such that a composition is obtained, preferably comprising no fibroblasts and/or keratinocytes (or less than 1000, 500, 50 fibroblasts and/or keratinocytes per mL (or per g). Preferably, the composition comprises less than 1000, 500, 200, 100, or 10 cells per mL (or per g). It is this composition that is the wound healing formulation according to the present disclosure. Preferably, at least 1, 2, 3, 4, 5 mL of the composition if provided, or at least 1, 2, 3, 4, 5 L of the composition is provided.

The composition obtainable by the method according to the present disclosure preferably is a pharmaceutical composition.

The method may thus further comprise step c) relating to providing a pharmaceutically acceptable formulation comprising the secretion obtained in step b), preferably in the form of e.g. a liquid, ointment, cream, gel, hydrogel, lotion, dressing, or patch. It is further preferred that the formulation comprises no remainder of cells, or less than 3, 2, 1, 0.5, 0.2, or 0.1 wt. % of such remainder.

In a particular embodiment, the composition is a (liquid) composition comprising

5-30 wt. %, preferably 10-20 wt. % CCL-2;

5-30 wt. %, preferably 15-25 wt. % CXCL-1;

35-65 wt%, preferably 45-55 wt. % CXCL-8;

0-2 wt. %, preferably 0.1-1 wt. % CCL-5;

1-5 wt. %, preferably 2-4 wt. % IL-6;

0-1 wt. %, preferably 0.005-0.1 wt. % IL-1α;

5-25 wt. %, preferably 5-15 wt. % TIMP-2;

0-3 wt. %, preferably 0.5-1.5 wt. % VEGF; and

0-2 wt. %, preferably 0.01-0.2 wt. % HGF,

relative to the total weight of said compounds in the composition, and wherein the composition preferably is an (aqueous) pharmaceutical composition. It is preferred that the above constituents relate to the human naturally occurring variants. The liquid composition can be the culture medium (as used in the method) including the wound healing mediators, which can be ready for use for wound healing purposes.

The above wound healing mediators can be quantified in a composition via e.g. ELISA, for example as according to the following Table:

TABLE 1
Details of antibodies that can be used for ELISA. Antibodies are
obtainable from R&D Systems Inc. (Minneapolis, MN, USA), except
for CXCL-8/IL-8 and TNF-α for which a Pelipair reagent set can
be used, obtainable from Sanquin (Amsterdam, the Netherlands). The
“Coating” and “Biotin” columns refer to the amounts of free
antibody and biotinylated antibody typically applied, where 1:100
refers to the dilution of the commercially available concentration.

| Antibody | Coating | Biotin |
|---|---|---|
| against | (μg/ml) | (μg/ml) |
| CCL-2/MCP-1 | 1.5 | 0.05 |
| CXCL-1/GRO-α | 0.5 | 0.2 |
| CXCL-8/IL-8 | 1:100 | 1:100 |
| CCL-5/RANTES | 0.5 | 0.01 |
| IL-6 | 2 | 0.2 |
| IL-1a | 2 | 0.0125 |
| TIMP-2 | 2 | 0.05 |
| VEGF | 1 | 0.05 |
| HGF | 1 | 0.4 |
| CCL-20 | 1 | 0.05 |
| TNF-α | 1:100 | 1:100 |

In general, each constituent, i.e. each wound healing mediator (or an active variant thereof), can be administered in a concentration of 0.001 pg/mL to 1000 μg/mL; preferably in the range of 0.1 pg/mL to 10 μg/mL, yet more preferably in the range of 1 pg/mL to 1000 ng/mL, even more preferably in the range of 10 pg/mL to 500 ng/mL.

Particularly preferred is a liquid composition comprising

40000-130000 pg/mL, preferably 90000-120000 pg/mL CCL-2;

30000-175000 pg/mL, preferably 120000-170000 pg/mL CXCL-1;

200000-400000 pg/mL, preferably 320000-375000 pg/mL CXCL-8;

100-3000 pg/mL, preferably 1500-2500 pg/mL CCL-5;

8000-25000 pg/mL, preferably 15000-25000 pg/mL IL-6;

0-200 pg/mL, preferably 50-150 pg/mL IL-1α;

50000-110000 pg/mL, preferably 45000-75000 pg/mL TIMP-2;

0-12000 pg/mL, preferably 5000-10000 pg/mL VEGF; and

150-600 pg/mL, preferably 200-500 pg/mL HGF,

or a composition wherein said concentration ranges are divided by 2, 3, 4, 5, 6, 7, 8, 9, 10, 15, or 20, so as to account for dilutions of the secretion obtainable by the present method.

The composition according to the present disclosure preferably contains no fibroblasts and/or keratinocytes (or less than 1000, 500, 100, 50, or 10 fibroblasts and/or keratinocytes per mL (or per g))

The composition may be in the form of a liquid, ointment, cream, gel, hydrogel, lotion, or dressing. The composition may further comprise pharmaceutically acceptable carriers. Such carriers can be formulated using any means known in the art (e.g. Remington's Pharmaceutical Sciences 16th edition (1980)). The composition may advantageously be used for topical administration onto a wound bed as to stimulate the healing of the wound. Administration of the composition thus may be via any mode of administration, but is preferably topical, such as by means of a liquid, aerosol, ointment, cream, gel, lotion, dressing or artificial dermal scaffold. It may also be applied by means of a patch comprising the composition.

In a further preferred embodiment, the composition is in the form of a (aqueous) solution, an ointment, a salve, a balsam, a tincture, an elixir, a plaster, a bandage, a dressing material, an alginate dressing, a topical solution, an infusion, or a surgical rinse solution. Sustained release preparations may be also be prepared and are foreseen by the present disclosure. Suitable sustained release preparations are e.g. described in WO97/03692, WO96/40072, WO96/07399, and U.S. Pat. No. 5,654,010, and may thus comprise the present composition.

Of course, additional constituents may be present in the composition. In this regard, the present inventors have previously described a full thickness skin tissue substitute and a method for the preparation thereof (WO/2005/068614). This skin substitute demonstrated good healing of inert, therapy resistant chronic wounds (with good scar formation) in comparison to full thickness autograft. The composition according to the present disclosure may be used in combination with this full thickness skin tissue substitute (such as comprised therein), e.g. for the treatment of wounds, particularly chronic wounds.

The present composition can be applied for medical use, preferably for use in the treatment of a wound (chronic or acute), preferably a skin wound, more preferably an ulcer, burn, surgical or decubitus wound. Ulcers are known to the skilled person and may refer to venous ulcers, diabetic (ulcers), and/or pressure ulcers. Decubitus ulcers/wounds (i.e. pressure sores or bedsores) refer to lesions caused by unrelieved pressure to any part of the body, especially portions over bony or cartilaginous areas.

The present composition can be used for closing a wound. Wound closure is the process of regenerating the covering cell layers of a tissue. Promoting wound closure means creating a positive effect in the regeneration of the covering cell layers. The positive effect can be an acceleration of the process or a decrease of the damaged area of the wound.

The treatment of a wound may comprise application of the composition onto the wound, preferably followed by application of a dressing, compression therapy, or application of a tissue substitute or an autograft onto the wound.

The wound may be pre-treated before application of the composition according to the present disclosure. Such pre-treatment may e.g. be washing out of the wound. In an embodiment, treatment of a wound comprises application of the composition onto the wound, followed by application of a dressing, compression therapy, vacuum persistent closure, or application of a tissue substitute or an autograft onto said wound. Of course, it is highly preferred that the composition is for topical administration. Application onto the site of injury (i.e. the wound) seems most advantageous.

The present composition may also be used to treat skin, for skin care or cosmetic use, e.g. for anti-ageing, i.e. to reduce progress of wrinkle formation, however the present composition is preferably not used for such purposes.

Methods of carrying out the conventional techniques used in methods of the present disclosure will be evident to the skilled worker, and are disclosed for example in Molecular Cloning: A Laboratory Manual (eds. Sambrook, J. & Russell, D. W.; Cold Spring Harbor Laboratory Press, Cold Spring Harbor, N.Y., USA, 2001).

### BRIEF DESCRIPTION OF THE FIGURES

FIG. 1. Flow diagram of production of the Wound Healing Formulation (WHF)

FIG. 2A. A fibroblast scratch assay shows a remarkable reduction in scratch size after 2 and 3 days with a 10% dilution of the WHF as compared to longEGF (1 ng/ml) alone and the negative control. Briefly, primary fibroblasts were cultured in a monolayer until 70% confluency, followed by a 4-days serum free culture period. A scratch was made which represents a wound. After wounding different culture conditions were added: 10% WHF TERT fib/A431 (75%/25%) supernatant (filled squares), longEGF (1 ng/ml) (dotted line) and a negative control (CKC1 medium only without supplements)(open squares). After 2 days the cultures were refreshed with similar culture conditions. Within the graph the relative reduction in scratch size is shown (n=4 donors, quadruplo, MEAN±SEM). The curves were statistically different as measured with a 2-way ANOVA followed by Tukey's multiple comparisons test.

FIG. 2B. Keratinocyte migration assay. Epidermal sheets from foreskin of 3 donors (duplo) were placed on a acellular donor dermis. After attachment the cultures were cultured air-exposed with different conditions (10% WHF, 30% WHF (both derived from TERT fib/A431 (75%/25%), longEGF (1 ng/ml) and medium only (neg. CKC2, including actrapid, isoprenaline and solucortef)) for approximately 2 weeks. The epidermal outgrowth was measured on histological H&E stained sections with NIS Elements. The graph shows a significant increased epidermal outgrowth which is stimulated by the WHF (10% and 30%). To check whether the longEGF is working, we added results from another experiment in which a higher concentration (2 ng/ml) and longer air-exposed culture period (3 wk) was used. As shown in Figure, after two weeks of air-exposed culture, the outgrowth of the epidermal sheets treated with 10% or 30% WHF was superior to longEGF (1 ng/ml) alone and the negative control. Data are represented as MEAN±SEM. Statistical analysis: one-way ANOVA; followed by Tukey's multiple comparisons test. * P<0.05; **P<0.01; ***P<0.001; ****P<0.0001

FIG. 3. Following the keratinocyte migration assay, H&E staining of the outgrowth shows that the epidermal outgrowth of the WHF supernatants (10% and 30%, derived from TERT fib/A431 (75%/25%)) indeed exhibits a better quality than the negative control and longEGF (1 ng/ml).

FIG. 4. Proliferation rate and viability of primary fibroblasts and keratinocytes was determined in the presence of 10% WHF, 30% WHF (both derived from TERT fib/A431 (75%/25%)), bFGF, EGF, or negative control. Briefly, primary fibroblasts (3.84*10⁴ cells/well, dy0 and dy4) or primary keratinocytes (2.24*10⁵ cells/well, dy0 and 2.0*10⁵ cells/well, dy3) were seeded into a 6-wells plate. Cells were counted with the Adam cell counter which also recorded the cell viability. Culture conditions: 10% and 30% WHF supernatant (TERT fib/A431 (75%/25%)). Positive control for fibroblasts was bFGF (10 ng/ml) and for keratinocytes EGF (10 ng/ml) and normal medium was used as “negative” control. When the cells reached 70-80% confluency, they were passaged, counted and cultured again for the same period. The primary fibroblasts and keratinocytes derived from normal skin (n=2) and foreskin (n=1). As can be seen in the Figure, WHF (10% and 30%) does not significantly affect the proliferation and viability of both cell types, however there is a positive trend of the WHF. Values represent MEAN±SEM, 3 donors (duplo).

FIG. 5. Differences in wound healing potency of the secretion of the cell line co-cultures (75/25, i.e. (75% TERT fib/25% A431)) compared to the secretion of the primary cell counterpart derived from either foreskin or adult abdominal skin (75% primary dermal fibroblasts/25% primary epidermal keratinocytes). 24 hour supernatant ELISA (IL-6 and IL-8) measurements were performed for the cocultures. Values represent MEAN±SEM.

The following Examples illustrate different embodiments of the disclosure. Unless stated otherwise all recombinant DNA techniques are carried out according to standard protocols as described in e.g. Sambrook et al. (1989) Molecular Cloning: A Laboratory Manual, Second Edition, Cold Spring Harbor Laboratory Press; and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA.

### EXAMPLE 1

In order to determine the endogenous secretion of three different cell lines, i.e. A431 keratinocytes, TERT-keratinocytes and BJ5-ta fibroblasts, 24-hour supernatants were collected from 70-80% confluent monolayer cell line cultures for ELISA measurements, see Table 2 below (n=2-3 experiments, duplo). Cell lines were cultured within clinical grade medium (CKC1) and for the production of secretion for 24 hours in clinical grade DMEM/HamF12 (3:1) medium only.

TABLE 2
Secretion of the three different cell lines when cultured separately.
Concentration in pg/ml after 24 hours of culturing (MEAN ± SD),
ND = below detection limit.

| | A431 | TERT | |
|---|---|---|---|
| | keratinocytes | keratinocytes | BJ5-ta fibroblasts |
| CCL-2/ | ND | ND | 5.957 ± 1.712 |
| MCP-1 | | | |
| CXCL-1/ | 61.605 ± 63.374 | ND | 698 ± 21 |
| GROα | | | |
| CXCL-8/ | 24.168 ± 26.919 | 716 ± 161 | 2.227 ± 1.762 |
| IL-8 | | | |
| CCL-5/ | 1.198 ± 421 | ND | 34 |
| RANTES | | | |
| IL-6 | 129 | ND | 275 ± 215 |
| IL-1α | 173 ± 85 | ND | ND |
| TNF-α | ND | ND | ND |
| TIMP-2 | 11.537 ± 5.356 | 22.834 ± 2.566 | 157.043 ± 67.116 |
| VEGF | 53.018 ± 12.985 | 2518 ± 546 | 255 ± 68 |
| HGF | ND | ND | 1.732 |

### EXAMPLE 2

This example shows that the secretion of a co-culture of immortalized keratinocytes and immortalized fibroblasts is richer in wound healing mediators as compared to the secretion of the cell lines when cultured separately (as in Example 1), and also as compared to a full-thickness skin equivalent or excised skin.

Multiple immortalized keratinocyte cell lines were tested (i.e. A431 obtainable from ATCC, CRL-1555; N/TERT-1 obtainable from Rheinwald's lab; and NCTC2544 obtainable from Italy, Cell Culture center, Istituto Zooprofilattico sperimentale; BS CL143) in a co-culture with the TERT-immortalized fibroblast cell line BJ-5ta (obtainable from ATCC, CRL-4001), wherein different ratios between keratinocytes and fibroblasts were used. The combination of the A431 keratinocyte cell line and the TERT fibroblast cell line in a 25%/75% ratio resulted in the most potent secretion (Wound Healing Formulation, WHF). Below, the experimental procedure and results are described in more detail.

Human Cell Line Skin Equivalent

To establish the interaction between the keratinocytes and fibroblasts, first a human skin equivalent was successfully constructed from immortalized keratinocytes and immortalized fibroblasts. This full-thickness cell line skin equivalent consisted of a well-differentiated epidermis on top of a fibroblast populated dermis. A haematoxylin-eosin staining of the skin equivalent showed a stratified epidermal layer with a stratum basale, stratum spinosum, stratum granulosum and stratum corneum. Within the dermal layer the TERT fibroblasts are present and produce their own matrix. A basement membrane is formed by interaction between the TERT immortalized keratinocytes and fibroblasts.

This skin equivalent was able to secrete a reasonable amount of multiple wound-healing mediators (like IL-6; CXCL-1/IL-8; CCL-2/MCP-1; CXCL-1/GRO-α; RANTES/CCL-5; VEGF; TIMP-2 (Table 3 below).

Co-Culture

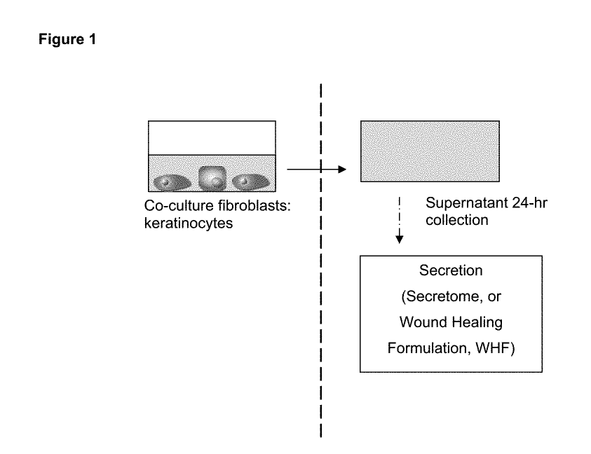

Surprisingly, a more simplified cell culture model (see FIG. 1), i.e. a co-culture of both immortalized keratinocytes and immortalized fibroblasts in a monolayer resulted in substantially higher wound-healing mediator levels than the secretion of a full-thickness skin equivalent (Table 3). Multiple immortalized keratinocyte cell lines were tested (A431, TERT keratinocytes and NCTC2544) in a co-culture with TERT-immortalized fibroblasts using different ratios.

The cell lines co-cultures were first cultured within clinical grade medium until 70% confluency was reached. For secretion production, the co-cultures were then cultured for 24 hours in clinical grade DMEM/HamF12 (3:1) medium only (obtainable from e.g. Sigma Aldrich).

After collection of 24 hour supernatant, ELISA measurements were performed on the secretions of different subsets of cultures. The human cell line equivalent was able to produce a cocktail of wound-healing mediators. In comparison with the ex vivo skin, the protein levels were either similar, increased or decreased. However, the co-cultures resulted in a much more enriched mixture of wound-healing mediators. Both the combination of TERT fibroblasts/A431 (75%/25%) and TERT fibroblast/TERT keratinocytes (75%/25%) resulted in a very potent wound-healing mediator cocktail, especially the combination of TERT fibroblasts (75%) and A431 keratinocytes (25%), see Table 3. Values are represented as MEAN±SD. ND=below detection limit.

TABLE 3
Secretion of the two different cell line cocultures is richer in chemokines, cytokines
and proteases & growth factors as compared to secretion of ex vivo skin, or secretion of a
human cell line skin equivalent. Concentration in pg/ml after 24 hours of culturing
(MEAN ± SD), ND = below detection limit.

| | | Cell line skin equivalent | Co-culture | Co-culture |
|---|---|---|---|---|
| | | TERT fib/TERT kera | TERT fib/TERT kera | TERT fib/A431 |
| | Ex vivo skin | (R&D medium) | (75/25) | (75/25) |
| CCL-2/MCP-1 | 1.497 ± 1.124 | 15.945 ± 7.442 | 64.333 ± 18.457 | 107.451 ± 51.257 |
| CXCL-1/GROα | 3.109 ± 1.689 | 35.439 ± 17.009 | 55.901 ± 36.781 | 153.556 ± 113.443 |
| CXCL-8/IL-8 | 19.647 ± 13.512 | 22.655 ± 12.774 | 250.964 ± 138.051 | 349.466 ± 146.296 |
| CCL-5/RANTES | 83 ± 40 | 108 ± 16 | 148 ± 61 | 2.061 ± 843 |
| IL-6 | 131.756 ± 64.057 | 568 ± 315 | 13.987 ± 3.617 | 18.924 ± 4.741 |
| IL-1α | 22 ± 15 | ND | ND | 82 |
| TNF-α | ND | ND | ND | ND |
| TIMP-2 | 38.355 ± 32.576 | 35.010 ± 5.846 | 93.238 ± 36.037 | 65.148 ± 18.320 |
| VEGF | 2.425 ± 499 | ND | ND | 7.011 ± 793 |
| HGF | 6.227 ± 4.209 | ND | 447 | 308 |

Upscaling from the regular T75 culture flasks towards a Nunc Factory system with 2 layers, suitable for high yield and large scale production with lot-to-lot consistency, was successful. Since the co-culture TERT fibroblasts in combination with A431 resulted in the most potent cocktail, it was decided to proceed with those two cell lines. Direct filtering (before −20° C. storage) of the supernatant with a 0.22 μm filter appeared to have no effect on the protein concentration.

### EXAMPLE 3

This example describes the differences in wound healing potency of the secretion of different co-cultures, wherein different ratios between fibroblasts and keratinocytes were applied. As described earlier, the 24 hour supernatant ELISA measurements were performed not only on different co-cultures, i.e. TERT fibroblasts either combined with A431 or TERT keratinocyte cell line, but also on cocultures having different ratios of fibroblasts/keratinocytes (i.e. 0/100; 25/75; 50/50; 75/25; 100/0). The results thereof are shown in Tables 4 and 5.

Most enriched was the secretion of a co-culture comprising 75% TERT fibroblasts and 25% A431 or TERT keratinocytes (Table 4). As already described in Example 1 and 2, the cell lines co-cultures were first cultured within clinical grade medium (CKC1) until they reached 70% confluency. For secretion production, the cell line co-cultures were cultured for 24 hours in clinical grade DMEM/HamF12 (3:1) medium only.

TABLE 4
Secretion of coculture of TERT fibroblasts and A431 keratinocytes in different ratios.
Concentration in pg/ml after 24 hours of culturing (MEAN ± SD), ND = below detection limit.

| | Ratio: | Ratio: | Ratio: | Ratio: | Ratio: |
|---|---|---|---|---|---|
| | 0/100 | 25/75 | 50/50 | 75/25 | 100/0 |
| CCL-2/MCP-1 | ND | 7.945 ± 8.313 | 42.488 ± 43.570 | 107.451 ± 51.257 | 6.151 ± 1.758 |
| CXCL-1/GROα | 61.605 ± 63.374 | 98.511 ± 87.228 | 120.028 ± 90.241 | 153.556 ± 113.443 | 732 |
| CXCL-8/IL-8 | 24.168 ± 26.919 | 119.176 ± 59.049 | 265.165 ± 17.477 | 349.466 ± 146.296 | 1.860 ± 1.480 |
| CCL-5/RANTES | 1.198 ± 421 | 2.284 ± 730 | 2.548 ± 1.001 | 2.061 ± 843 | 68 |
| IL-6 | 129 | 2.517 ± 1.715 | 8.270 ± 2.659 | 18.924 ± 4.741 | 267 ± 197 |
| IL-1α | 173 ± 85 | 119 ± 34 | 87 ± 66 | 82 | ND |
| TNF-α | ND | ND | ND | ND | ND |
| TIMP-2 | 11.537 ± 5.356 | 25.058 ± 10.379 | 37.710 ± 17.754 | 65.148 ± 18.320 | 170.096 ± 77.132 |
| VEGF | 53.018 ± 12.985 | 42.614 ± 8.541 | 27.099 ± 4.988 | 7.011 ± 793 | 261 ± 46 |
| HGF | ND | ND | ND | 308 | 1.865 |

TABLE 5
Ratio TERT fibroblasts/TERT keratinocytes and resulting concentration wound
healing mediators in the secretion (pg/ml) after 24 hours of culturing (MEAN ± SD), ND = below
detection limit.

| | Ratio: | Ratio: | Ratio: | Ratio: | Ratio: |
|---|---|---|---|---|---|
| | 0/100 | 25/75 | 50/50 | 75/25 | 100/0 |
| CCL-2/MCP-1 | ND | 12.221 ± 10.075 | 33.018 ± 24.192 | 64.333 ± 18.457 | 5.763 ± 1.666 |
| CXCL-1/GROα | ND | 30.804 ± 42.189 | 50.580 ± 59.793 | 55.901 ± 36.781 | 664 ± 42 |
| CXCL-8/IL-8 | 716 ± 161 | 95.121 ± 73.078 | 223.312 ± 54.284 | 250.964 ± 138.051 | 2.593 ± 2.043 |
| CCL-5/RANTES | ND | 147 ± 19 | 150 ± 45 | 148 ± 61 | ND |
| IL-6 | ND | 2.787 ± 2.201 | 9.258 ± 4.768 | 13.987 ± 3.617 | 283 ± 233 |
| IL-1α | ND | ND | ND | ND | ND |
| TNF-α | ND | ND | ND | ND | ND |
| TIMP-2 | 22.834 ± 2.566 | 46.476 ± 20.943 | 73.923 ± 26.893 | 93.238 ± 36.037 | 143.991 ± 57.100 |
| VEGF | 2518 ± 546 | 563 | ND | ND | 248 ± 89 |
| HGF | ND | ND | ND | 447 | 1.598 |

### EXAMPLE 4

This example describes different wound healing assays that were performed. The secretion (Wound Healing Formulation, WHF) derived from the TERT fibroblast/A431 (75%/25%) co-culture was tested in different in vitro test models using primary fibroblasts and keratinocytes from different donors as proof of principle.

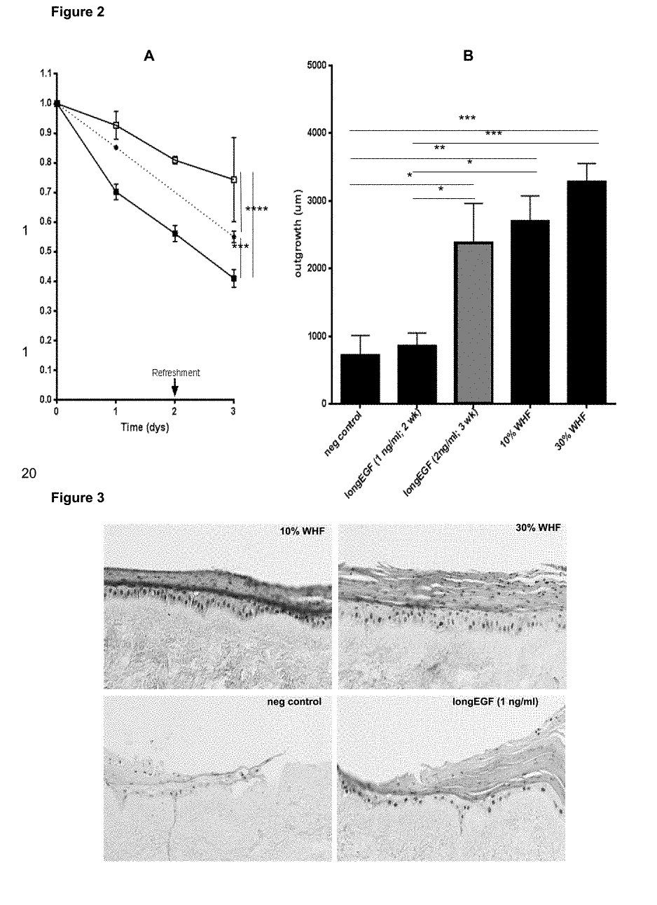

First, the effect of the WHF was tested on the migration of fibroblasts. Within a fibroblast scratch assay a remarkable reduction in scratch size was observed after 2 and 3 days with a 10% dilution of the WHF as compared to longEGF (1 ng/ml) alone and the negative control (FIG. 2A). Briefly, Primary fibroblasts were cultured in a monolayer until 70% confluency, followed by a 4-days serum free culture period. A scratch was made which represents a wound. After wounding different culture conditions were added: 10% WHF TERT fib/A431 (75%/25%) supernatant (filled squares), longEGF (1 ng/ml) (dotted line) and a negative control (CKC1 medium only without supplements)(open squares). After 2 days the cultures were refreshed with similar culture conditions. Within the graph the relative reduction in scratch size is shown (n=4 donors, quadruplo, MEAN±SEM). The curves were statistically different as measured with a 2-way ANOVA followed by Tukey's multiple comparisons test.

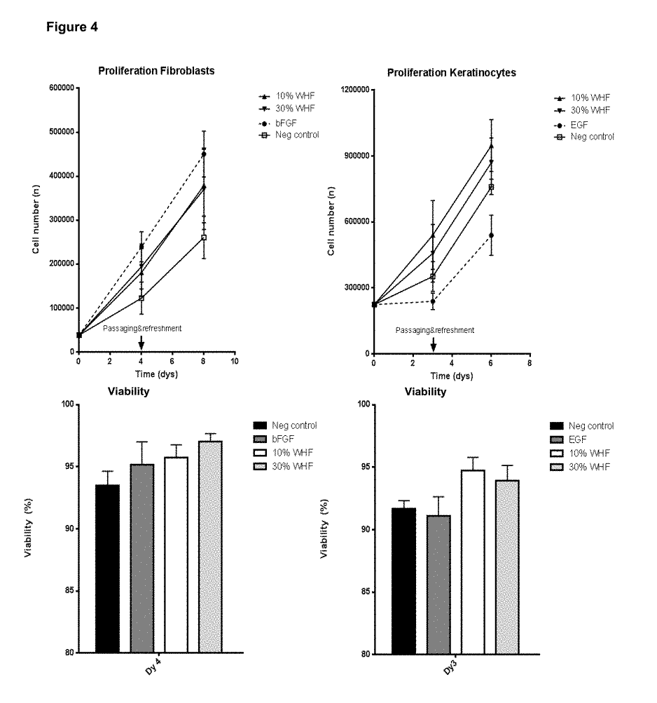

Secondly, the effect of the WHF on the migration behaviour of keratinocytes was established (FIG. 2B). Briefly, the epidermal sheets from foreskin of 3 donors (duplo) were placed on a acellular donor dermis. After attachment the cultures were cultured air-exposed with different conditions (10% WHF, 30% WHF, longEGF (1 ng/ml) and medium only (CKC2, including actrapid, isoprenaline and solucortef) for approximately 2 weeks. The epidermal outgrowth was measured on histological H&E stained sections with NIS Elements. The graph shows a significant increased epidermal outgrowth which is stimulated by the WHF (10% and 30%). To prove that the longEGF is working, we added results from another experiment in which a higher concentration (2 ng/ml) and longer air-exposed culture period (3 wk) was used. As shown in FIG. 2B, after two weeks of air-exposed culture, the outgrowth of the epidermal sheets treated with 10% or 30% WHF was superior to longEGF (1 ng/ml) alone and the negative control. In addition, the quality of the restored epidermis was better than long EGF (1 ng/ml) and the negative control. As can be seen in FIG. 3, the H&E staining of the outgrowth shows that the epidermal outgrowth of the WHF supernatants (10% and 30%) indeed exhibits a better quality than the negative control and longEGF (1 ng/ml).

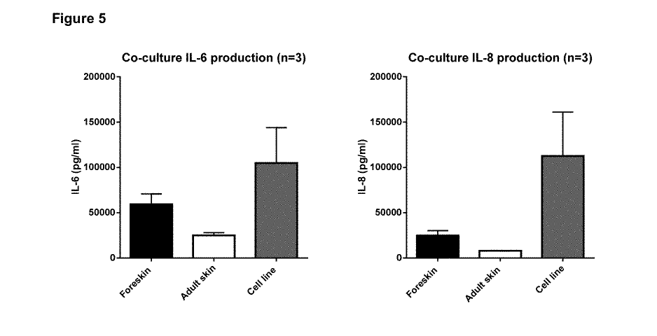

Thirdly, the effect of the WHF on normal proliferation rate and viability of primary fibroblasts and keratinocytes was determined. Briefly, primary fibroblasts (3.84*10⁴ cells/well, dy0 and dy4) or primary keratinocytes (2.24*10⁵ cells/well, dy0 and 2.0*10⁵ cells/well, dy3) were seeded into a 6-wells plate. Cells were counted with the Adam cell counter which also recorded the cell viability. Culture conditions: 10% and 30% WHF supernatant (TERT fib/A431 (75%/25%)). Positive control for fibroblasts was bFGF (10 ng/ml) and for keratinocytes EGF (10 ng/ml) and normal medium was used as “negative” control. When the cells reached 70-80% confluency, they were passaged, counted and cultured again for the same period. The primary fibroblasts and keratinocytes derived from normal skin (n=2) and foreskin (n=1). As can be seen in FIG. 4, WHF (10% and 30%) does not significantly affect the proliferation and viability of both cell types, however there is a positive trend of the WHF. Values represent MEAN±SEM, 3 donors (duplo).

In summary, within a fibroblast scratch assay, a 10% dilution of WHF showed to reduce scratch size more than long EGF (1 ng/ml) alone. Within a keratinocyte migration assay, 10% and 30% dilutions of WHF stimulated epidermal outgrowth more as compared to longEGF (1 ng/ml) alone. In addition, WHF does not affect proliferation and viability of the primary fibroblasts and primary keratinocytes used in the assays. In conclusion, the WHF is able to improve wound-closure as shown by the in vitro migration studies, while maintaining the normal proliferation rate and viability of the cells.

### EXAMPLE 5

A coculture of immortalized keratinocytes and immortalized fibroblasts can produce a richer secretion as compared to a coculture of primary keratinocytes and primary fibroblasts. This example describes the differences in wound healing potency of the secretion of the immortalized cell line co-cultures (75/25) compared to the secretion of the primary cell counterpart derived from either foreskin or adult abdominal skin (75% primary dermal fibroblasts/25% primary epidermal keratinocytes). As described earlier, 24 hour supernatant ELISA (IL-6 and IL-8) measurements were performed for the cocultures (see FIG. 5)

Notably, most enriched was the secretion of a co-culture comprising 75% TERT fibroblasts and 25% A431 (cell line). As already described in Example 1 and 2, the co-cultures were first cultured within clinical grade medium (CKC1) until they reached 70% confluency. For secretion production, the co-cultures were cultured for 24 hours in clinical grade DMEM/HamF12 (3:1) medium only.

1. Method for producing a therapeutic wound healing composition, wherein the method comprises the steps of:
a) co-culturing immortalized fibroblasts and immortalized keratinocytes, thereby producing secretion;
b) separating the secretion from the fibroblasts and keratinocytes; and
c) providing a therapeutic wound healing composition comprising the secretion.

2. Method according to claim 1, wherein in step a) the percentage of the immortalized fibroblasts relative to the total number of immortalized fibroblasts and immortalized keratinocytes is between 5% and 95%.

3. Method according to of claims 1, wherein
the immortalized fibroblasts are TERT-immortalized fibroblasts; and/or
the immortalized keratinocytes are A431 keratinocytes or TERT-immortalized keratinocytes.

4. Method according to claims 1, wherein step a) is performed
in vitro;
at 30-40° C.;
for at least 1 hour; and/or
in a clinical grade medium.

5. Method according to claim 1, wherein in step a)
the immortalized fibroblasts and immortalized keratinocytes are arranged randomly in the co-culture; and/or
the immortalized fibroblasts and immortalized keratinocytes are co-cultured together in a monolayer of cells.

6. A composition obtainable by the method according to claim 1, wherein the composition is a pharmaceutically acceptable composition.

7. A method for the treatment of a wound, comprising applying the composition of claim 6 to the wound in an amount effective for the treatment of the wound.

8. The method according to claim 7, wherein the treatment of a wound comprises applying the composition onto a skin wound.

9. The composition according to claim 6, wherein the composition contains no fibroblasts and/or keratinocytes.

10. The composition according to claim 6, wherein the composition is in the form of a liquid, ointment, cream, gel, hydrogel, lotion, dressing, or carrier.

11. Method according to claim 1, wherein co-culturing immortalized fibroblasts and immortalized keratinocytes, thereby producing secretion, provides a percentage of the immortalized fibroblasts relative to the total number of immortalized fibroblasts and immortalized kerotinocytes between 25% and 95%.

12. Method according to claim 2, wherein
the immortalized fibroblasts are TERT-immortalized fibroblasts; and/or
the immortalized keratinocytes are A431 keratinocytes or TERT-immortalized keratinocytes.

13. Method according to claim 4, wherein co-culturing immortalized fibroblasts and immortalized keratinocytes, thereby producing secretion, is performed at 34-38° C.

14. Method according to claim 4, wherein co-culturing immortalized fibroblasts and immortalized keratinocytes, thereby producing secretion, is performed for at most 240 hours.

15. Method according to claim 1, wherein co-culturing immortalized fibroblasts and immortalized keratinocytes, thereby producing secretion, is performed
in vitro;
at 34-38° C.;
for 1 hour to 240 hours; and
in a clinical grade medium.

16. The method of claim 8, wherein the applying the composition onto a skin wound is followed by applying a dressing, compression therapy, or a tissue substitute or an autograft onto the skin wound.
